(19) United States
(12) Patent Application Publication
(10) Pub. No.: US 2020/0008836 A1
(43) Pub. Date: Jan. 9, 2020

(54) LOCALIZATION AND CHARACTERIZATION OF SUBSURFACE STRUCTURES USING TEMPORALLY-RESOLVED PHOTON DENSITY WAVES

(71) Applicant: The Regents of the University of California, Oakland, CA (US)

(72) Inventors: Bruce J. Tromberg, Irvine, CA (US); Jesse Lam, Irvine, CA (US); Timothy Quang, Irvine, CA (US)

(21) Appl. No.: 16/452,248

(22) Filed: Jun. 25, 2019

Related U.S. Application Data
(60) Provisional application No. 62/694,689, filed on Jul. 6, 2018.

(51) Int. Cl. A61B 17/34; A61B 8/00

(52) U.S. Cl. CPC A61B 17/3403; A61B 2017/3413; A61B 8/466

(57) ABSTRACT

Optical systems and methods to track the position of a needle in subsurface structures, such as tissues or organs, and co-register the information with ultrasound are described herein. An optical fiber in a needle catheter is used to transmit light inside of the structure. The light is intensity modulated at sufficiently high frequencies such that the time of arrival of the light can be used to determine the distance of the needle from an optical detector at the tissue surface. The position of the needle can be tracked by combining data obtained using different modulation frequencies and/or wavelengths of light. By using multiple detectors at different positions, the location of the needle in 3D space can be triangulated using light, and the data can be integrated with ultrasound to obtain the anatomical structure.

### CROSS-REFERENCES TO RELATED APPLICATIONS

This application is a non-provisional, and claims benefit of U.S. Provisional Patent Application No. 62/694,689, filed Jul. 6, 2018, the specification of which is incorporated herein in its entirety by reference.

### GOVERNMENT SUPPORT

This invention was made with Government support under Grant No. P41EB015890, awarded by the National Institutes of Health and Grant No. FA9550-17-0193, awarded by the U.S. Air Force Office of Scientific Research. The Government has certain rights in the invention.

### FIELD OF THE INVENTION

The present invention relates to systems and methods for tracking and localizing subsurface tissue probes, such as needles, and also characterizing subsurface objects, such as tumors, using temporally-resolved diffuse photon density waves.

### BACKGROUND OF THE INVENTION

Precise tracking and localization of thin needles buried in turbid tissues is difficult using ultrasound technology due to lack of contrast. In a typical clinical procedure, ultrasound imaging is used to identify anatomical structures and to guide the needle insertion to deliver a treatment to a target. However, visualizing the needle on the ultrasound is a persistent challenge and complicates the procedure.

There have been some developments that attempt to address this issue. For instance, Cha et al. discloses a needle with optically illuminated tip that may help localization of the needle in turbid tissue (Cha W, Ro J H, Wang S G, et al. Development of a device for real-time light-guided vocal fold injection: A preliminary report. Laryngoscope. 2016; 126(4):936-940. doi:10.1002/lary.25661). This previously explored technique utilized line-of-sight detection (i.e. human vision) and intensity information to localize the needle, thus quantitative positional information is unavailable. Attenuation of the light can be the result of many factors including needle depth and tissue optical properties, making it difficult to accurately track the needle location in thick turbid tissues and thereby limiting the Cha technique to applications involving thin, homogeneous, or relatively transparent tissues. Hence, there exists a need for new technologies that can track, localize, and characterize needles and subsurface objects in any type of tissue.

The present invention features an optical method to track the position of a needle catheter deep inside of tissue and co-register the information with ultrasound. This method uses an optical fiber in a needle catheter to transmit light inside of a tissue. The light is intensity modulated at sufficiently high frequencies, typically MHz or higher, such that the time of arrival of the light can be used to determine the distance of the needle from an optical detector at the tissue surface. The position of the needle can be tracked by combining data obtained using different modulation frequencies and/or wavelengths of light. By use of multiple detectors at different positions, the location of the needle in 3-D space can be triangulated, and the data can be integrated with ultrasound. Thus, the anatomical structure is obtained using ultrasound, and the position of the needle is obtained using light.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

It is an objective of the present invention to provide for systems and methods to of tracking a needle in subsurface structures. Embodiments of the invention are given in the dependent claims. Embodiments of the present invention can be freely combined with each other if they are not mutually exclusive.

According to some aspects, the present invention utilizes intensity-modulated light emitting from an optical fiber within a needle or catheter in order to track a position of the needle or catheter in biological tissue. Several wavelengths of light (e.g. by use of different laser diodes) at one or more modulation frequencies may be used to increase sensitivity to the needle position. The light can be detected using a sufficiently sensitive photodetector, such as an avalanche photodiode, photomultiplier tube, or silicon photomultiplier placed on the outside surface of the tissue under investigation. The modulated light, which needs to be at high enough frequencies in order measure the temporal dispersion between the needle and the detector, e.g. megahertz-gigahertz, produces photon density waves, which can be detected by frequency or time domain methods.

In some embodiments, if frequency domain detection is utilized, the amplitude and phase of the detected light can be used to determine the position of the detector since phase encodes time, which is proportional to distance through the phase velocity of the photon density wave. In some instances, when the light source is closest to the detector (i.e. the linear distance between the tip of the emitting fiber and the detection active area is shortest) amplitude will be at the peak. This is because there are the fewest attenuators (absorbers and scatterers) at this point. At the same time, as there are the fewest amount of scatterers between the light source and detector, the phase delay is at its minimum.

In other embodiments, if time domain detection is utilized, amplitude may also be used in a similar manner and the photon time-of-flight (TOF) is measured directly by recording to temporal point spread function. At the thinnest part of the sample at which the source and detector are minimally separated, the photon time-of-flight will be shortest. Various computational methods can be used to analyze the TOF curve in order to develop a distance metric. Thus, by using time domain or frequency domain photon migration, a direct correlation relating amplitude, phase (or time-of-flight), and position of the needle can be derived, and the position of the needle can be calculated.

According to some embodiments, single or multiple frequencies and single or multiple wavelengths may be utilized to improve sensitivity and information content. One or more photo detectors at the surface can be used to solve for the position of the needle in N-dimensional space. Once the position of the needle is known, the data can be integrated with ultrasound data, providing real-time guidance of the needle or catheter to an ultrasound identified target. This may be helpful when determining the location of the needle is critical to the success of a procedure. For example, this method may be used to help guide clinicians to particular targets inside tissue, such as for delivering anesthetic drug to a specific nerve site or guiding the needle to a biopsy site. This method has specific advantages when the needle is difficult to visualize and track using ultrasound. In addition, this method for delivery of light deep in tissue to a subsurface object or structure can be used to characterize specific features of that buried object or target tissue such as the object/structure's optical absorption, scattering, and physiological properties.

One of the unique and inventive technical features of the present invention is the use of frequency domain photon migration (FDPM) methods to extract a position of the needle, allowing a procedure to be performed with greater precision. Without wishing to limit the invention to any theory or mechanism, it is believed that the technical feature of the present invention advantageously provides for sub-millimeter resolution tracking of a needle in turbid tissue. This technique may also allow a procedure (e.g. injection of drug into specific tissue sites) to be performed more safely, as it eliminates reliance on “eye-balling” by providing quantitative positional information of the needle in biological tissue. Finally, the present technique allows for characterization of the optical and physiological properties of the object, which may change upon administration of therapy via the needle and could be used as feedback for therapeutic guidance and dosing. By using amplitude and phase information acquired by FDPM, this technique will provide sub-millimeter resolution tracking of a needle in turbid tissue. Due to the nature of near-infrared light, this method is appropriate for use in turbid tissue up to several centimeters deep. Trilateration of the needle is also possible, and can be integrated with ultrasound, allowing physicians to simultaneously access physiological information, as well as the position of the needle in the ultrasound field of view. None of the presently known prior references or work has the unique inventive technical feature of the present invention.

Furthermore, the prior references teach away from the present invention. For example, one having ordinary skill in the art would not expect photon detection-based techniques to allow for determination of the pathlength in turbid media or non-transparent tissue. The reason is light scattering in turbid medium or non-transparent tissue. Such light scattering results in a tortious pathlength, which is not the desired simple direct distance between source and detector. Here, despite the light scattering in the turbid media, the present invention allows for a quantitative measurement of a simple direct distance between source and detector. Previous transillumination methods relied on direct visualization of light output from the optical fiber; as the light source progresses closer to the tissue surface, the intensity of the light should be brighter, and the spot should increase in size. However, these are only subjective measures of the actual position of the light source and provide no objectively quantifiable data on the light sources relative position. Furthermore, this approach only applies to situations in which the light source is being advanced towards the tissue surface. In the case of the present invention, the location of the light source can be quantified regardless if the light source is being advanced toward or away from the surface.

Furthermore, the inventive technical features of the present invention contributed to a surprising result. For example, while it is surprising that a light based technique allows for any determination of distance in a turbid medium, it is especially surprising that the technique allows for position determination with sub-millimeter accuracy because other light based techniques can only provide generally qualitative determinations of location. Without wishing to limit the present invention to any particular theory or mechanism, it is believed that use of near-infrared light (600-1000 nm) allows for probing of tissue on the order of centimeters. As a non-limiting example, the use of wavelengths outside this range may only allow for detection of signals on the order of micrometers to millimeters.

### BRIEF DESCRIPTION OF THE DRAWINGS

This patent application contains at least one drawing executed in color. Copies of this patent or patent application publication with color drawings will be provided by the Office upon request and payment of the necessary fee.

The features and advantages of the present invention will become apparent from a consideration of the following detailed description presented in connection with the accompanying drawings in which:

FIG. 1A shows a non-limiting schematic of a system of the present invention.

FIG. 1B shows a flowchart illustrating the algorithm used to determine the distance between a modulated light source and a detector.

FIG. 2 shows another schematic of the system.

FIG. 3 shows a non-limiting example of a temporally modulated light source creating PDWs, which are attenuated in the tissue. The attenuated light can be detected, where the detected signal is diminished in amplitude and delayed in phase with respect to the source (Bruce J. Tromberg, Lars O. Svaasand, Tsong-Tseh Tsay, and Richard C. Haskell, “Properties of photon density waves in multiple-scattering media,” Appl. Opt. 32, 607-616 (1993)).

FIG. 4A shows an experimental setup using the system of the present invention, where the fiber optic is inserted within the catheter needle.

FIG. 4B shows a closer view of the experimental setup in FIG. 4A, illustrating the placement of the fiber optic within the needle catheter.

FIG. 5 shows amplitude times phase metric in a one-dimensional scan. The peak of the response corresponds to the point in which the source fiber was directly in front of the detector. The traces demonstrate differences in response for different wavelengths and different modulation frequencies.

FIGS. 6A-6B show recovered signals from highlighted detectors in a top view depicting detector positions. FIG. 6A shows phase response from two detectors symmetrically across from the needle tip. FIG. 6B shows phase response from three detectors that are collinear with the needle trajectory.

FIG. 7A-7B show reconstructed needle positions from a top-down view (FIG. 7A) and a side view (FIG. 7B) of the phantom geometry. The black arrow represents the needle trajectory, blue diamonds represent the location of the detectors, and the asterisks represent the computed needle tip coordinates. The black circle represents the approximate dimensions of the buried target.

FIG. 8A shows the schematic layout of the needle, detectors, and target and the calculation of the known distances between the needle and the detectors.

FIG. 8B shows the calibration of the system by selecting any two points on each curve to determine the phase/distance relationship for each detector.

FIG. 8C shows example calculations for the phase to distance conversion using the known phase/distance pairs and an unknown point.

FIG. 8D shows an illustration of the trilateration calculation used to determine the position of the unknown point by solving the system of equations given by three detectors.

FIG. 8E shows the calculated position of the probe based on a fitting of the data.

### DESCRIPTION OF PREFERRED EMBODIMENTS

As used herein, the term “turbid” refers to a medium, tissue, fluid, or environment which is non-transparent, cloudy, opaque, or thick with suspended matter. As a non-limiting example, tissue of a mammal may be considered to be turbid.

According to some embodiments, the present invention features a system that includes the following components: a small-diameter optical fiber, a needle catheter, a laser with laser driver, photodetectors, and an analyzer instrument to measure the timing features of the light that propagates through the tissue. The needle catheter is used to guide the optical fiber to the site of interest and the optical fiber is used to guide light to the tissue. The laser and laser driver can deliver the light to the optical fiber. The analyzer instrument provides the radio-frequency signal to modulate the laser, as well as analyze the frequency response of the tissue. The photodetectors can detect the light in the tissue and transmits the corresponding signals to a computer, which calculates the position of the needle. In order to calculate the position of the needle in three-dimensional space, software is needed in order to perform three fundamental tasks: 1) acquire amplitude and phase from the light injected in the tissue; 2) calculate the three-dimensional position of the needle tip; and 3) co-register and display the data with ultrasound. In some embodiments, the system can be used in conjunction with ultrasound to obtain structural information. Since fine needle catheters are difficult to identify using ultrasound alone, this invention can be used to triangulate and overlay the needle position on the ultrasound.

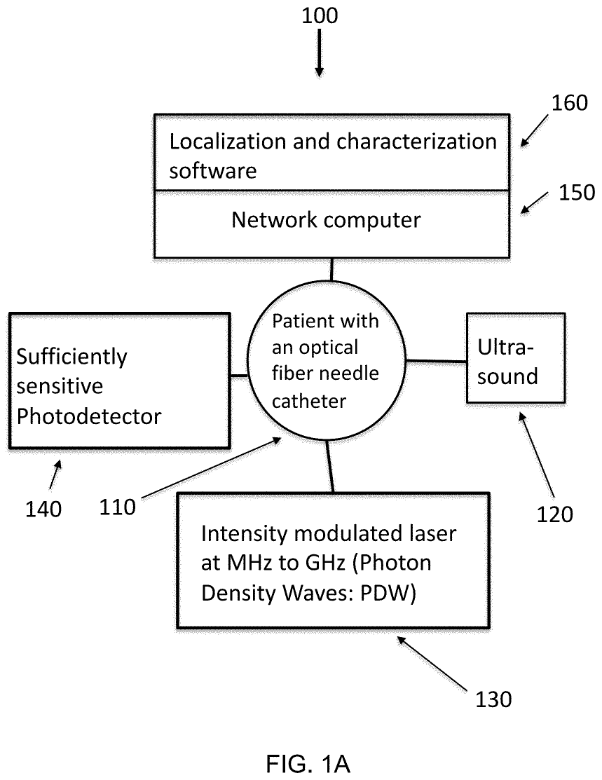

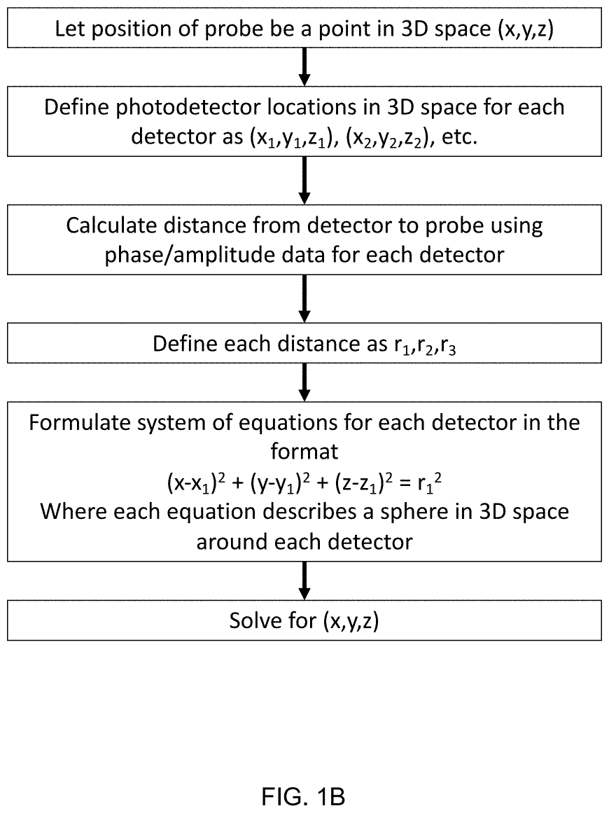

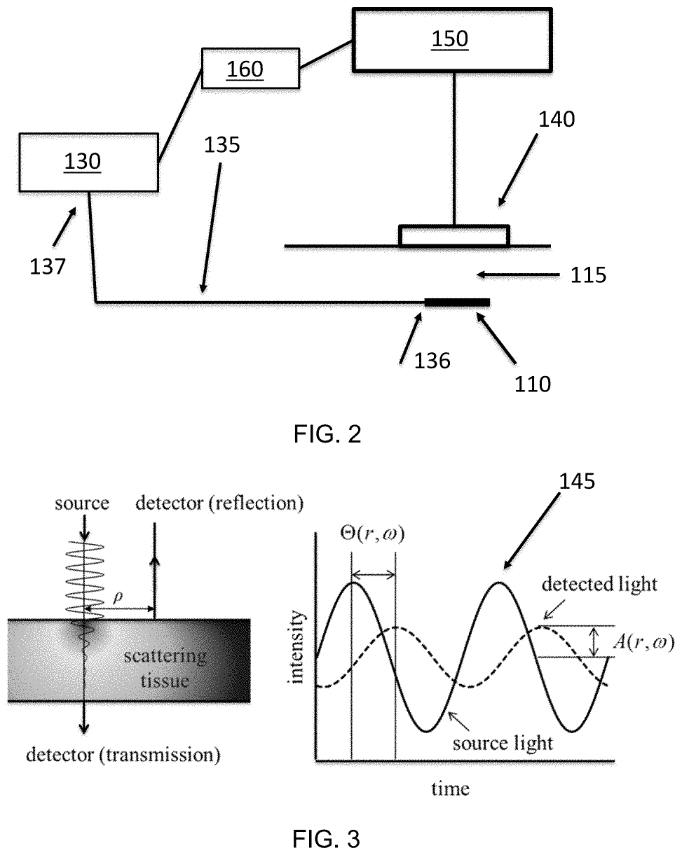

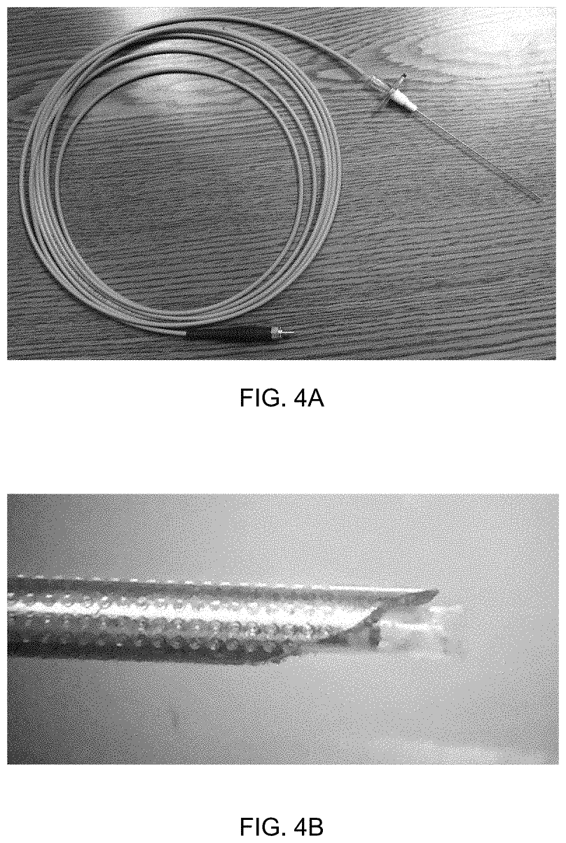

Referring now to FIGS. 1-3, in some embodiments, the present invention features a system (100) of localization and characterization of a subsurface object in a turbid medium using a temporally-resolved photon density wave (PDW) (145) to quantitatively determine a distance between the subsurface object and a photodetector (140). The system may comprise a needle catheter (110), a fiber optic (135) with a first end (136) embedded in the needle catheter (110) and a second end (137) operatively connected to a laser device (130), the laser device (130) emitting a light with light intensity modulated at MHz to GHz to generate the PDW (145), and the photodetector (140) effective to detect the PDW (145). In one embodiment, the PDW (145) is configured to pass through the fiber optic (135), be emitted from the needle catheter (110), and be detected by the photodetector (140). In another embodiment, detection of the PDW (145) is configured to allow for localization of the needle catheter (110) by quantitative determination of a distance (148) between the second end (137) of the fiber optic (135) and the photodetector (140).

In other embodiments, the light intensity may be modulated at a frequency lower than MHz or higher than GHz. As a non-limiting example, the light intensity may be modulated a frequency of about 500 kHz, 600 kHz, 700 kHz, 800, kHz, 900 kHz, 1 MHz, 100 MHz, 200 MHz, 300 MHz, 400 MHz, 500 MHz, 600 MHz, 700 MHz, 800 MHz, 900 MHz, 1 GHz, 100 GHz, 200 GHz, 300 GHz, 400 GHz, 500 GHz, 600 GHz, 700 GHz, 800 GHz, 900 GHz, 1 THz, 100 THz, or higher than 100 THz. As an additional non-limiting example, the light intensity may be modulated in a range of about 50 MHz to 1 GHz. In one embodiment the emitted light may have a wavelength in the near-infrared region. As a non-limiting example, the emitted light may have a wavelength in the range of about 600-610, 610-620, 620-630, 630-640, 640-650, 650-660, 660-670, 670-680, 680-690, 690-700, 700-710, 710-720, 720-730, 730-740, 740-750, 750-760, 760-770, 770-780, 780-790, 790-800, 800-810, 810-820, 820-830, 830-840, 840-850, 850-860, 860-870, 870-880, 880-890, 890-900, 900-910, 910-920, 920-930, 930-940, 940-950, 950-960, 960-970, 970-980, 980-990, or 990-1000 nm.

In some embodiments, the system (100) may additionally comprise an ultrasound device (120) to generate ultrasound at or near the needle catheter (110), and a computer (150) with subsurface object localization and characterization software (160). In some preferred embodiments, the software (160) may comprise a set of instructions that, when executed by the computer (150), causes the computer to perform operations to computationally track the needle catheter (110) movement derived from the PDW, register local tissue mapping information relative to an organ (115) derived from the ultrasound (120), and calculate the needle catheter (110) location relative to the organ (115). Once the movement of the needle catheter (110) relative to the organ (115) is tracked via PDW (145), a data information of the needle movement derived from PDW (145) can be integrated with the local tissue mapping information relative to the organ (115) derived from the ultrasound (120), thereby providing a real-time guidance of needle catheter to an ultrasound identified target.

According to some embodiments, at least one photodetector may be used at the surface to detect the position of the needle in N-dimensional space. Non-limiting examples of the photodetector (140) include an avalanche photodiode, a photomultiplier tube, and a silicon photomultiplier placed on an outside surface of the tissue to study the organ (115). In one embodiment, the PDW (145) may be detected via the photodetector (140) by a frequency domain. In another embodiment, the PDW (145) may be detected via the photodetector (140) by a time domain detection.

In some embodiments, multiple laser devices (130) can be used, and at least one intensity modulation frequency can be used to increase sensitivity to the needle position. The modulated light can be at a high frequency in the order of megahertz to gigahertz to measure the temporal dispersion between the needle and the detector. In some embodiments, system is configured to allow for a quantitative localization of the needle catheter (110) with at least millimeter or at least sub-millimeter accuracy.

According to other embodiments, the present invention also features a method of localizing and characterizing a subsurface probe in a turbid tissue using a temporally-resolved photon density wave (PDW) to quantitatively determine a distance between the subsurface probe and one or more photodetectors. In one embodiment, the method may comprise operatively coupling one or more laser devices to an optical fiber, coupling the optical fiber to the subsurface probe, inserting the subsurface probe into a tissue, and positioning the photodetectors externally to the tissue and in a vicinity of the subsurface probe. In another embodiment, the method includes advancing the subsurface probe towards a target site and illuminating the tissue by emitting a light from the laser devices to the optical fiber. The light intensity of the light may be modulated by the laser devices to generate the PDW. The method may further comprise detecting, via the photodetectors, signals comprising amplitude and phase data corresponding to changes in the PDW scattered back from the tissue and transmitting said signals to the detected light to a computer, and calculating a position of the subsurface probe using a trilateration algorithm to obtain physical coordinates of the probe by quantitative determination of a distance between the probe and the photodetectors. The amplitude and phase data are inputted into a trilateration algorithm to determine the physical coordinates of the probe. An ultrasound device may be used to obtain an ultrasound image of the target site and surrounding tissue and the physical coordinates of the probe can be co-registered and displayed with the ultrasound image.

In one embodiment, the present invention features a method of identifying a position of a probe in a turbid or non-transparent medium using a temporally-resolved photon density wave (PDW) to quantitatively determine a distance between the probe and one or more photodetectors. As a non-limiting example, the method may comprise: operatively coupling one or more laser devices to an optical fiber; coupling the optical fiber to the probe; inserting the subsurface probe into the turbid medium; positioning the photodetectors in a vicinity of the probe; emitting a light from the laser devices through the optical fiber, wherein an intensity of the light is modulated by the laser devices to generate the PDW; detecting, via the photodetectors, signals corresponding to changes in the PDW due to scattering by the turbid medium, wherein the signals comprise amplitude and phase data; and calculating a position of the probe to obtain physical coordinates of the probe, wherein the amplitude and phase data are used to determine the physical coordinates of the probe.

According to some embodiments, the photodetectors may be positioned external to the turbid medium. According to some other embodiments, the position of the probe may be calculated using a trilateration algorithm. In one embodiment, the method may allow for a quantitative identification of the position of the probe with at least millimeter, or at least sub-millimeter resolution. In another embodiment, the method may additionally include a separate imaging technique and co-registry of the position of the probe with an image from the separate imaging technique. Non-limiting examples of separate imaging techniques include ultrasound, x-ray, MRI, CT, OCT, photoacoustic, and echocardiography imaging techniques.

### EXAMPLE

The following is a non-limiting example of an optical method to track the position of a needle catheter inside of tissue. It is to be understood that said example is not intended to limit the invention in any way, and that equivalents or substitutes are within the scope of the invention.

Frequency domain photon migration (FDPM) is an optical technique that illuminates tissue with high-frequency (e.g. 50 MHz to 600 MHz) intensity-modulated near-infrared lasers and detects changes in the light scattered back from the tissue to determine tissue optical properties. When frequency domain detection is utilized, the amplitude (A) and phase shift (φ) of the detected light can be used to determine the position of a light source delivered inside the needle tip using a thin optical fiber. This is due to the fact that the phase shift (φ) of the intensity modulated light is linearly proportional to the distance (d) between the light source (i.e. the needle tip) and the detector, where d=φC/ω, C is the velocity of light in the medium (c/n), and ω is the angular modulation frequency.

In multiple scattering tissues, the phase is related to the actual distance the scattered light travels between the source and detector. This value is greater than and proportional to the mean free path or direct “line of sight” linear distance. As a result, when the light source is closest to the detector (i.e. the linear distance between the tip of the emitting fiber and the detection active area is shortest) amplitude (A) will be at the peak and phase (φ) will be at its lowest. The present invention utilizes FDPM to track the needle position in order to meet the following aims: 1) obtain preliminary data tracking needle position in one-dimension and develop an optical index for tracking the position as a function of phase and frequency, 2) develop a method to track needle position in two-dimensions, and 3) develop a method to track needle position in three-dimensions.

### Experimental Design

### Data Acquisition

For each experiment, the laser diode sources from an FDPM system were coupled to a 400 μm core optical fiber which was then inserted into an 18 gauge hypodermic needle. The source fiber was then attached to a linear translation stage which advanced the fiber along a straight line in regular intervals. The majority of the testing was performed in a gelatin-based optical phantom; optical properties were adjusted to simulate tissue scattering properties such that the needle was visually obscured. In each experiment, the source fiber was advanced in fixed intervals while FDPM data was collected at each step. An external avalanche photodiode (APD) was fixed in place on the surface of the phantom to detect the scattered light from the source fiber at every position. The advancing needle was controlled by a motorized linear-stage. The APD was placed externally and in contact with the top surface of the phantom. The phantom was constructed such that the embedded needle was not visible. In the first experiment, the needle was inserted 2 cm beneath the surface of the phantom and translated toward a buried object, which was a 1 cm diameter grape, simulating a nerve plexus 2 cm deep and 6 cm from the needle entry point.

Initial experiments focused on tracking the signal from the FDPM source using a single detector to characterize the FDPM response to changes in needle position. In the next set of experiments, a new set of measurements were acquired, and additional detectors were included in order to recover the needle position in multiple dimensions (2D and 3D). A total of 12 detector positions and 501 modulation frequencies from 100 MHz to 600 MHz were tested to provide a more comprehensive dataset to illustrate the positional dependence of the detector response, evaluate the frequency dependent resolution, and to test different detector arrangements in order to optimize detector placement.

### Reconstructing Needle Position

The approach to reconstructing the needle tip position was comprised of two steps: 1) exploring the relationship between FDPM phase and absolute distance and 2) application of a trilateration algorithm to obtain physical coordinates of the needle tip. Trilateration is a technique with practical applications in navigation, namely global positioning systems. In the first step, it was assumed that the starting position of the needle tip and the distance at which the needle tip is closest to the detector are both known. Based on these two points, the phase readings were converted into distances. Next, the distance data of all the detectors used is inputted into a trilateration algorithm which computes the needle tip coordinates in three dimensions.

### Calculating Distance

The distance between the FDPM source and a detector can be calculated as follows: 1) Assume that the starting position of the tip of the source and the distance at which the needle tip is closest to the detector are both known. 2) Convert phase into distance for each using the two known points 3) input distance data from all detectors into trilateration algorithm which computes needle tip coordinates in three dimensions. As a non-limiting example, the computation for the distance from probe to detector may be done computationally as a minimization problem using a trilateration algorithm and not as an analytical solution. For the phase to distance conversion, the following equation may be used:

$$d = \frac{{s}_{m} - {s}_{1}}{{s}_{2} - {s}_{1}} * ( {d}_{2} - {d}_{1} ) + {d}_{1}$$

where: Sₘ=measured signal
d₁,d₂=known distances
s₁, s₂=known signals, and
d=distance from probe to detector

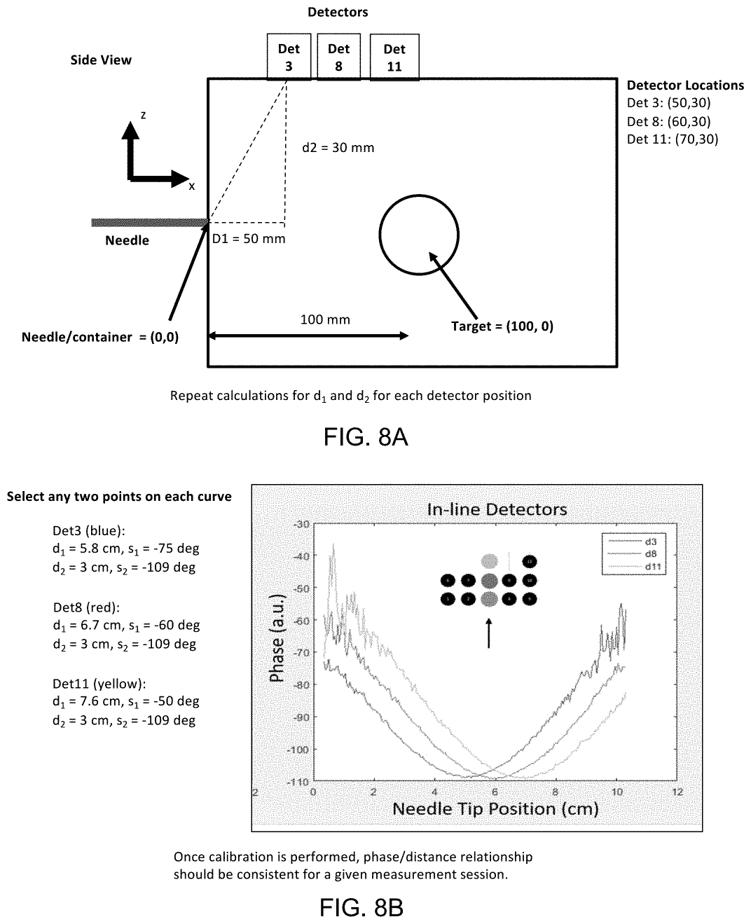

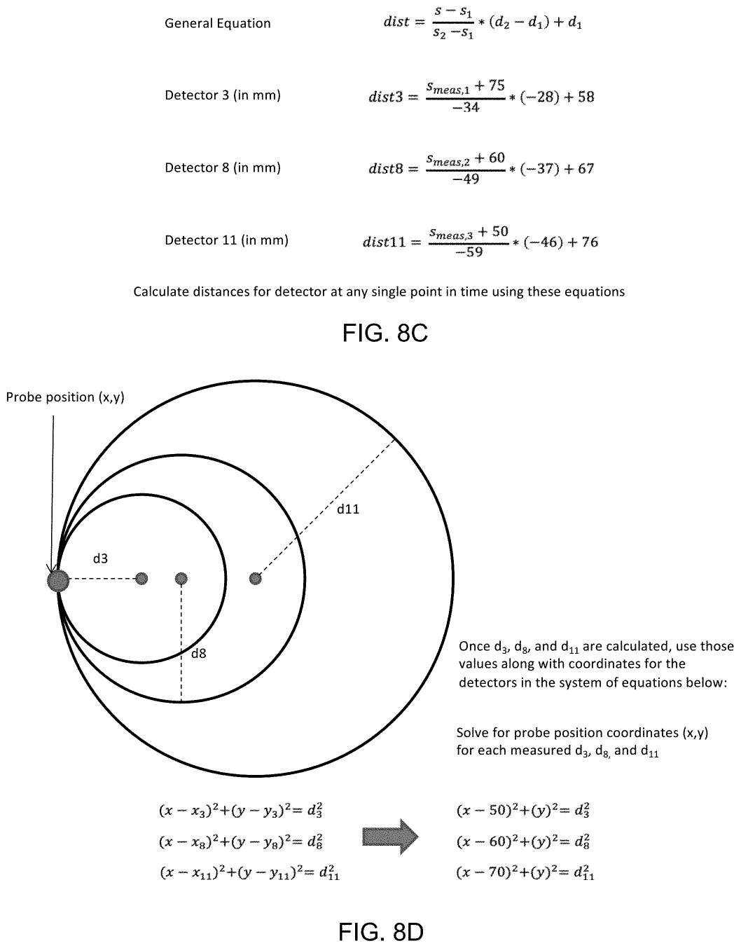

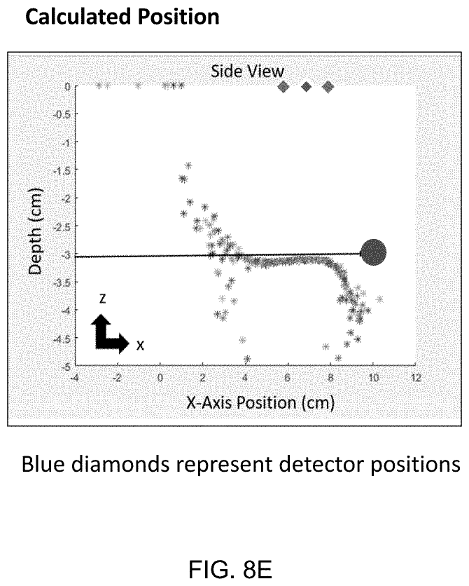

FIGS. 8A-8E provide an example of a 2D calculation of needle position using the trilateration algorithm. First, calibration of the system must be done by moving the needle to at least two positions for which the distance to each detector may be calculated. The signal or phase is measured at each of the two positions so as to provide at least two known distance/signal pairs for each detector. Having thus calibrated the system, the location of the probe may be determined in any unknown position using the general equation provided in FIG. 8C. A distance from the unknown position to each detector is calculated using the formula and the two known distance/signal pairs for the corresponding detector. Given the calculated distances to each detector and the known coordinates of each detector, the system of equations provided in FIG. 8D allow for determination of the probe position by solving for probe position coordinates (x, y). The calculated position allows for a plotting of the fitted data, as shown in FIG. 8E.

### Results

Without wishing to limit the present invention to a particular theory or mechanism, it is believed that the phase would be at its minimum and amplitude would be at its maximum at the point where the fiber was directly in front of the APD.

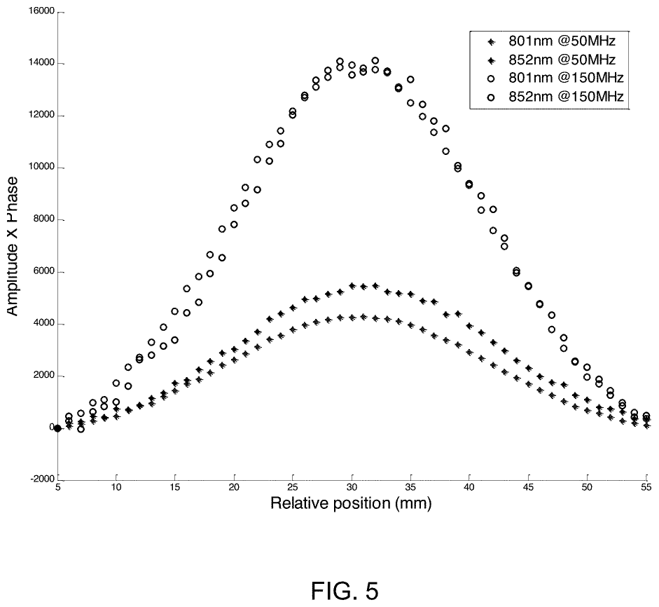

FIG. 5 shows the detector responses from the one-dimension experiments with two different laser wavelengths and two different modulation frequencies. Preliminary results from the one-dimensional experiments supported that the peak signal would occur at the point in which the source fiber was directly in front of the detector. Higher frequencies may yield better positional resolution (˜125 units/mm, 50 MHz vs. ˜750 units/mm, 150 MHz) at the expense of signal to noise ratio. These data showed that spatial resolution depends on the modulation frequency and signal-to-noise ratio and may range from about 0.5-1.0 mm.

In the next set of experiments, additional detectors were incorporated in order to reconstruct the needle tip's position in two and later three dimensions. Differences in the detector responses were observed based on where the detectors were placed on the phantom. The data shown in FIG. 5 were acquired from 12 different detector locations to reconstruct the needle tip position in three dimensions.

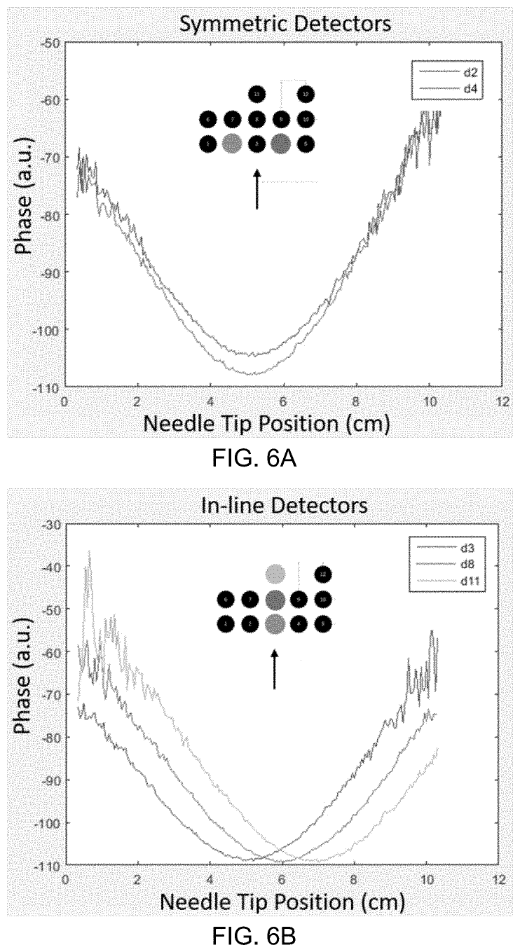

FIG. 6A shows the phase response from two symmetrically placed detectors; the insert denotes where the detectors are in relation to the needle. A phase “trough” occurs when the linear distance between the tip of the needle and the detector is minimized. In this second experiment, the needle was inserted 3 cm beneath the surface of the phantom and translated toward a buried object, the 1 cm diameter grape, simulating a nerve plexus 3 cm deep. The total needle travel to the buried object was 10 cm. The laser was modulated at 200 MHz. Theoretically, if the needle was exactly in between the two detectors, then the phase responses would overlap with each other. In this case, given that the responses do not overlap, in can be inferred that the needle is closer to detector 4 because of the greater response. FIG. 6B illustrates the phase from the three detectors that are collinear with the needle path. The acquired data supported that detector 3 reached its minima first as it is the first detector the needle passed under, followed by detectors 8 and 11.

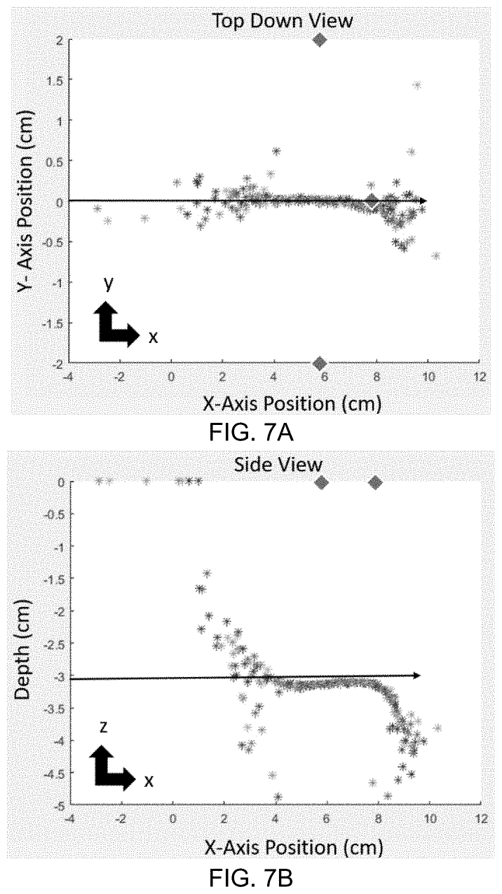

FIG. 7A shows the reconstructed needle position for one example detector arrangement from a top-down view of the phantom. FIG. 7B shows the reconstruction from a side view to show the performance of the reconstruction with regards to the Z-direction. When the needle is within 4 cm of the detector array, the reconstruction matches well with the theoretical trajectory of the needle.

The preceding example described an optical method for tracking the tip of a needle in three dimensions. Without wishing to limit the present invention, an approach utilizing FDPM has shown sufficient sensitivity to track the position of the embedded needle in turbid media. In some embodiments, a minimum of three detectors on the surface of the sample is recommended to recover three dimensional positions. In other embodiments, additional detectors beyond this minimum number may improve the accuracy of the reconstruction.

The range of the applied algorithm, that is, the maximum distance away from the detectors where position of the embedded needle can be confidently recovered, may be limited by the signal-to-noise ratio (SNR). However, this is not a technological limitation, but a result of hardware limitations. Small-area (1 mm), high speed detectors were utilized as the optimal modulation frequency had yet to be determined. However, results from the described experiments suggest that high-frequency (>200 MHz) modulation is not a necessity. In some embodiments, lower-speed, detector arrays may result in higher SNR since larger area, more sensitive detectors can be used. Detector arrays can have a small footprint and be lightweight which would be beneficial for any clinical implementation.

In some embodiments, the present technique can be used to deliver drugs directly to a nerve plexus; however, the invention is not limited to this application. Any clinical application which relies on needle based drug delivery or biopsy may utilize this approach. In other embodiments, the phase and amplitude information can be used to determine the properties of the tissue in the field of view of the needle. This information may be further used to identify the tissue type and complement the needle tracking data.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are solely for ease of examination of this patent application, and are exemplary, and not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A system (100) of localization and characterization of a subsurface object in a turbid medium using a temporally-resolved photon density wave (PDW) (145) to quantitatively determine a distance between the subsurface object and a photodetector (140), the system comprising:
a needle catheter (110);
a fiber optic (135) with a first end (136) and a second end (137), wherein the first end (136) is embedded in the needle catheter (110) and the second end (137) is operatively connected to a laser device (130);
the laser device (130) emitting an intensity modulated light to generate the PDW (145); and
the photodetector (140) effective to detect the PDW (145);
wherein the PDW (145) is configured to pass through the fiber optic (135), be emitted from the needle catheter (110), and be detected by the photodetector (140), and
wherein detection of the PDW (145) is configured to allow for localization of the needle catheter (110) by quantitative determination of a distance (148) between the second end (137) of the fiber optic (135) and the photodetector (140).

2. The system of claim 1, wherein the system additionally comprises:
an ultrasound device (120) to generate ultrasound at or near the needle catheter (110); and
a computer (150) with subsurface object localization and characterization software (160), wherein the software (160) comprises a set of instructions that, when executed by the computer (150), causes the computer to perform operations to computationally:
i. track the needle catheter (110) movement derived from the PDW;
ii. registering local tissue mapping information relative to an organ (115) derived from the ultrasound (120); and
iii. calculate the needle catheter (110) location relative to the organ (115);
wherein once the movement of the needle catheter (110) relative to the organ (115) is tracked via PDW (145), data information of the needle movement derived from PDW (145) can be integrated with the local tissue mapping information relative to the organ (115) derived from the ultrasound (120), thereby providing a real-time guidance of needle catheter to an ultrasound identified target.

3. The system of claim 1, wherein the PDW (145) is detected via the photodetector (140) by a frequency domain.

4. The system of claim 1, wherein the PDW (145) is detected via the photodetector (140) by a time domain detection.

5. The system (100) of claim 1, wherein multiple laser devices (130) are used, and at least one intensity modulation frequency is used to increase sensitivity to the needle position.

6. The system (100) of claim 1, wherein the photodetector (140) is an avalanche photodiode, photomultiplier tube, or silicon photomultiplier placed on an outside surface of the tissue to study the organ (115).

7. The system (100) of claim 1, wherein the modulated light is at a high frequency in the order of megahertz to gigahertz to measure the temporal dispersion between the needle and the detector.

8. The system (100) of claim 1, wherein at least one photodetector is used at a surface to detect a sub-surface position of the needle in N-dimensional space.

9. The system (100) of claim 1, wherein the system is configured to allow for a quantitative localization of the needle catheter (110) with at least millimeter accuracy.

10. A method of localizing and characterizing a subsurface probe in a turbid tissue using a temporally-resolved photon density wave (PDW) to quantitatively determine a distance between the subsurface probe and one or more photodetectors, said method comprising:
operatively coupling one or more laser devices to an optical fiber;
coupling the optical fiber to the subsurface probe;
inserting the subsurface probe into a tissue;
positioning the photodetectors externally to the tissue and in a vicinity of the subsurface probe;
advancing the subsurface probe towards a target site;
illuminating the tissue by emitting a light from the laser devices to the optical fiber, wherein an intensity of the light is modulated by the laser devices to generate the PDW;
detecting, via the photodetectors, signals corresponding to changes in the PDW scattered back from the tissue and transmitting said signals to the detected light to a computer, wherein the signals comprise amplitude and phase data; and
calculating a position of the subsurface probe using a trilateration algorithm to obtain physical coordinates of the probe, wherein the amplitude and phase data are inputted into a trilateration algorithm to determine the physical coordinates of the probe by quantitative determination of a distance between the subsurface probe and each photodetector.

11. The method of claim 10, wherein the method additionally comprises:
obtaining an ultrasound image of the target site and surrounding tissue; and
co-registering and displaying the physical coordinates of the probe with the ultrasound image.

12. The method of claim 10, wherein the PDW is detected via the photodetector by a frequency domain.

13. The method of claim 10, wherein the PDW is detected via the photodetector by a time domain detection.

14. The method of claim 10, wherein the photodetector is an avalanche photodiode, photomultiplier tube, or silicon photomultiplier.

15. The method of claim 10, wherein the modulated light is at a high frequency in the order of megahertz to gigahertz to measure the temporal dispersion between the subsurface probe and the photodetector.

16. A method of identifying a position of a probe in a turbid medium using a temporally-resolved photon density wave (PDW) to quantitatively determine a distance between the probe and one or more photodetectors, the method comprising:
operatively coupling one or more laser devices to an optical fiber;
coupling the optical fiber to the probe;
inserting the subsurface probe into the turbid medium;
positioning the photodetectors in a vicinity of the probe;
emitting a light from the laser devices through the optical fiber, wherein an intensity of the light is modulated by the laser devices to generate the PDW;
detecting, via the photodetectors, signals corresponding to changes in the PDW due to scattering and absorption by the turbid medium, wherein the signals comprise amplitude and phase data; and
calculating a position of the probe to obtain physical coordinates of the probe, wherein the amplitude and phase data are used to determine the physical coordinates of the probe by quantitative determination of a distance between the subsurface probe and the one or more photodetectors.

17. The method of claim 16, wherein the photodetectors are positioned external to the turbid medium.

18. The method of claim 16, wherein the position of the probe is calculated using a trilateration algorithm.

19. The method of claim 16, wherein the method allows for a quantitative identification of the position of the probe with at least millimeter resolution.

20. The method of claim 19, wherein the method additionally includes a separate imaging technique and co-registry of the position of the probe with an image from the separate imaging technique.
